(19) United States
(12) Patent Application Publication
(10) Pub. No.: US 2022/0161011 A1
(43) Pub. Date: May 26, 2022

(54) Transdermal Delivery System

(71) Applicant: Jeff Dotson, Bloomfield Township, MI (US)

(72) Inventor: Jeff Dotson, Bloomfield Township, MI (US)

(21) Appl. No.: 17/533,589

(22) Filed: Nov. 23, 2021

Related U.S. Application Data
(60) Provisional application No. 63/118,081, filed on Nov. 25, 2020.

(51) Int. Cl. A61M 35/00

(52) U.S. Cl. CPC A61M 35/10; A61M 11/042; A61M 2205/3653; A61M 2205/3673

(57) ABSTRACT

A transdermal delivery system: a delivery device; a band configured to connect the delivery device to a body part of a user, the delivery device comprising: a delivery chamber that is configured to receive a substance and a heating chamber configured to heat the substance in the delivery chamber so that the substance is liquified, vaporized, or both and exits the delivery chamber into contact with the body part of the user so that the substance is transdermally absorbed.

### CROSS-REFERENCE TO RELATED APPLICATION(S)

This application claims priority to and the benefit of U.S. Provisional Application Patent Ser. No. 63/118,081, filed Nov. 25, 2020, the entire disclosure of which is hereby incorporated by reference.

### FIELD

The present application relates to a device for liquifying or vaporizing a solid or a liquid and transferring the solid or liquid so that the solid or liquid is absorbed transdermally while minimizing leakage of any of the solid or liquid.

### BACKGROUND

Attempts have been made to deliver therapeutic drugs and/or pharmaceutical drugs to a user through the skin via a liquid application on the skin which may leak. These devices may be wearable such as a patch that is secured to a user's skin and then discarded after use.

Examples of delivery systems may be disclosed in U.S. Pat. Nos. 5,405,614; 6,113,940; or 10,232,156 all of which are expressly incorporated herein by reference for all purposes. Thus, there is a need for a delivery system that incorporates a solid substance and then delivers the solid substance so that the substance may be transmitted through the skin. There is a need for a delivery system that vaporizes a liquid or solid and then applies the vapor to a skin of a user. It would be desirable to have a delivery system that melts or softens a solid so that the solid may be transmitted through a user's skin.

### SUMMARY

The present teachings provide: a transdermal delivery system: a delivery device; a band configured to connect the delivery device to a body part of a user, the delivery device comprising: a delivery chamber that is configured to receive a substance and a heating chamber configured to heat the substance in the delivery chamber so that the substance is liquified, vaporized, or both and exits the delivery chamber into contact with the body part of the user so that the substance is transdermally absorbed.

The present teachings provide a delivery system that incorporates a solid substance and then delivers the solid substance so that the substance may be transmitted through the skin. The present teachings provide a delivery system that vaporizes a liquid or solid and then applies the vapor to a skin of a user. The present teachings provide a delivery system that melts or softens a solid so that the solid may be transmitted through a user's skin.

### BRIEF DESCRIPTION OF THE DRAWINGS

FIG. 1 is a perspective view of a transdermal delivery system.

FIG. 2 is a cross-sectional view of the delivery device of FIG. 1.

FIG. 3 illustrates a bottom view of a delivery device.

FIG. 4 is a cross-sectional view of the delivery device of FIG. 4.

FIG. 5 is a cross-sectional view of a perforation.

FIG. 6 is a cross-sectional view of a perforation.

FIG. 7 illustrates a flap blocking off a portion of the delivery device of FIG. 3.

### DETAILED DESCRIPTION

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the teachings. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. Other combinations are also possible as will be gleaned from the following claims, which are also hereby incorporated by reference into this written description.

The present teachings relate to a delivery system. The delivery system functions to provide a substance to a user. The delivery system function to provide a substance to skin of a user so that the substance may be transdermally absorbed into the skin of the user. Preferably, the delivery system functions to provide medication or cannabinoid to skin or a user so that the medication or cannabinoid may be absorbed transdermally. The delivery system may be in direct communication with a user's skin. The delivery system may be a watch, bracelet, necklace, connected to an arm, a leg, a wrist, a neck, a waist, or a combination thereof of a user. The delivery system may intermittently, continuously, or both provide a substance. The delivery system may contain the substance and maintain the substance within the delivery system until the user instructs the delivery system to apply the substance or until a predetermined setting is achieved and the substance is applied.

The substance may be any substance that may be transdermally absorbed. The substance may be a solid, wax, semi-solid, a liquid, a vapor, or a combination thereof. The substance may be melted, liquified, vaporized, or a combination thereof. The substance may be or include a medication, an-over the counter drug, a prescribed drug, cannabis, a cannabinoid, cannabidiol (CBD), or a combination thereof. The substance may be applied to skin of a user via a delivery device of the delivery system.

The delivery device functions to apply the substance unto skin (dermis) of a user. The delivery device may meter an amount of substance applied to a user. The delivery device may directly apply the substance to skin of the user. The delivery device may house the substance, heat the substance, apply the substance, self-contain the substance, and maintain the substance on the skin without the substance leaking. The delivery device may include an open space that includes one or more chambers, two or more chambers, three or more chambers, or four or more chambers. The delivery device may include an open space that includes ten or less chambers, eight or less chambers, or six or more chambers. The delivery device may have a housing that contains a plurality of chambers and is connected to a user via one or more attachment mechanisms.

The housing functions to contain the elements of the delivery system and to deploy all or a portion of the substance to the user. The housing functions to contain a power source, electrical elements, control elements, or a combination thereof. The housing may be sufficiently large to contain enough components to deliver the substance, to house 8 hours of substance or more, 12 hours of substance or more, or even 24 hours of substance or more. The housing may hold 1 week of substance or less, 5 days of substance or less, or 3 days of substance or less. The housing may be sufficiently small to fit on a wrist of a male, a female, or both. The housing may be made of or include plastic, metal, a conductive material, a porous material, a closed material, a material that may not be damaged when heated, a material that may not transmit heat to a user, or a combination thereof. The housing may be round, square, rectangular, octagonal, pentagonal, triangular, a geometric shape, a symmetrical shape, an asymmetrical shape, or a combination thereof. The housing may have a thickness of about 1 cm or more, 2 cm or more, 3 cm or more, 4 cm or more, 4.5 cm or more, or about 5 cm or more. The housing may have a thickness of about 10 cm or less, 7 cm or less, or 6 cm or less. The housing may have a thickness of between about 1 cm and 5 cm or about 2 cm and 4 cm. The housing may be held in contact with a user's skin via an attachment mechanism.

The attachment mechanism functions to hold the delivery device in contact with skin, on a user, in a predetermined location, or a combination thereof. The attachment mechanism may create a temporary connection, a removable connection, a reusable connection, a disposable connection, or a combination thereof. The attachment mechanism may include an adhesive. The attachment mechanism may be a band, a necklace, tape, a clasp, hook and loop fastener, lock, a elastomeric, two pieces that hook together, once piece that hooks onto itself, one continuous loop that locks onto itself, two way tape, adhesive, or a combination thereof. The attachment mechanism may be similar to a watch or bracelet. The attachment mechanism may connect the delivery device to a user and maintain a connection with the user so that a substance in the delivery chamber is moved into contact with the user upon a predetermined condition being met.

The delivery chamber functions to hold all or a portion of the substance and assist in delivering some or all of the substance to a user. The delivery chamber may be a chamber or a door of a chamber that may open and close. The delivery chamber may house the substance, a solid, wax, semi-solid, liquid, vapor, or a combination thereof. The delivery chamber may seal a substance therein until a predetermined time is achieved and the delivery chamber may release the substance. The delivery chamber may be in direct contact with a user's skin. The delivery chamber may include a porous surface that is juxtaposed with the user's skin. The delivery chamber may prevent solids, liquids, semi-solids, waxes or a combination thereof from exiting the delivery chamber. The delivery chamber may allow vapors to exit the delivery chamber. The delivery chamber may have a portion that is movable between an open position and a closed position. The delivery chamber may be connected to one or more distribution members.

The one or more distribution members function to distribute the substance to skin or a user. The distribution members may transport the substance to the skin of a user. The distribution members may hold the substance in contact with a user's skin. The distribution members may be porous, have through holes, channels, or a combination thereof. The distribution members may be made of the same material as the housing. The distribution member may allow a liquid to extend through, a vapor extend through, or both. The distribution member may include a solid portion, a porous portion, or both. The distribution member may funnel the substance from one opening to an area larger than the opening. The distribution member and the opening may have a ratio of size. The distribution member may distribute to an area that is about 2:1 or more, 3:1 or more, 4:1 or more, or about 5:1 or more an area of the opening. The distribution member may distribute to an area of about 10:1 or less, 8:1 or less, or about 7:1 or less an area of the opening. The distribution member may be a malleable substance that may create contact with the housing and skin. The distribution member may be an elastomeric substance, a sponge like substance, collagen, a biocompatible substance, a substance that may be replaceable, a substance that may absorb some or all of the substance that is delivered and assist in holding the substance in contact with skin, or a combination thereof. The distribution member may extend out of the delivery chamber. The distribution member may receive substance when the heater within the heater chamber is active and the substance is liquified, vaporized, or both and released from the delivery chamber. The distribution member may include one or more perforations.

The perforations function to permit the substance to extend out of the delivery system into contact with a user. The perforations may be a channel (e.g., extend from a top of the distribution member to a bottom of the distribution member. The perforations may be a through hole on a bottom of the distribution member. The perforations may permit the substance to pass through. The perforations may permit only a gas or vapor to pass therethrough and may restrict a liquid from passing through. The perforations may be randomly spaced as placed within the distribution member. The perforations may be equally distributed across an area of the distribution member. The perforations may be changed depending upon a type of substance being applied. The perforations may be openable and closeable to adjust a rate the substance is applied. For example, a flap may be moved to cover some or all of the perforations. The flap may be rotated or slid into place to cover some of the perforations. The flap may be external to the delivery system. The flap may be located within the delivery system and may be controlled by rotating a portion of the delivery system or sliding a slider of the delivery system.

The perforations may be manmade. The perforations may be naturally occurring. For example, a portion of the distribution member may be made of a porous material. The perforations may have a continuous area from a top to a bottom. The perforations may vary in area from a top to a bottom. For example, a top of the perforations may be larger than a bottom so that the top may receive the substance and channel the substance to the bottom (e.g., conical). The perforations may be a combination of different sizes and shapes. Some or all of the perforations may be conical, continuous, cylindrical, or a combination thereof. The perforations may include material that assists in retaining the substance in contact with the user's skin. For example, cotton, linen, collagen, a sponge, or a combination thereof may be within the perforations. The substance may be maintained between the delivery system and skin of a user by one or more sealing members retaining the substance as the substance extends out of the perforations of the distribution member.

The sealing members function to maintain a substance on a user's skin, between the delivery device and the user's skin, or both. The sealing member may prevent the substance from leaking or moving outside of an area of the delivery device. The sealing member may create a periphery that prevents vapors, liquids, semi-solids, or a combination thereof from exiting an area bounded by the sealing member, the delivery device, the delivery chamber, or a combination thereof. The sealing member may be a rigid material, a soft material, an elastomeric material, a rubber material, a closed cell material, a compressible material, or a combination thereof. The sealing member may mirror a shape of the delivery device. The sealing member may extend around a periphery of the delivery device. The sealing member may be circular, square, rectangular, octagonal, pentagonal, hexagonal, or a combination thereof. The sealing member may be one continuous piece. The sealing member may have a portion that extends into the delivery chamber. The sealing member may surround the distribution member. The sealing member may have a height. The height of the sealing member and the height of the distribution member may be substantially equal. The height of the sealing member may be slightly greater than a height of the distribution member so that the distribution member does not interfere with sealing. The distribution member and the sealing member may have substantially equal heights. The distribution member and the sealing member may both be compressible. The sealing member may be compressed so that the distribution member may be moved into contact with skin. The sealing member may be compressed by the attachment mechanism. The attachment mechanism may compress the sealing member against skin of a user to form a seal. The sealing member may form a seal so that when the heating chamber heats, liquefies, vaporizes, or a combination thereof the substance, the substance is maintained with an area of the delivery device.

The heating chamber functions to deliver heat to the substance. The heating chamber may liquify the substance, vaporize the substance, cause the substance to flow, or a combination thereof. The heating chamber may be sealed from the delivery chamber. The heating chamber may transmit heat to the delivery chamber. The heating chamber may include one or more heaters, one or more thermal transfer areas, or both. The heating chamber may circulate heat from the heating chamber into the delivery chamber. The heating chamber may heat air and move the heat from the heating chamber into the delivery chamber. The heating chamber may provide indirect heat, direct heat, or both. The heating chamber may provide a combination of heat and vibration. The heating chamber may agitate the substance to assist in vaporizing or liquifying the substance. The heating chamber may apply ultrasonic vibrations, may shake the substance, may impinge the substance with air, or a combination thereof. The substance may be heated, agitated, or both so that the substance moves from the heating chamber.

The heater functions to change a state of the substance. The heater may change the substance from a solid to a semi-solid, a liquid, a vapor, or a combination thereof. The heater may directly heat the substance, indirectly heat the substance, heat by convection, heat by conduction, heat by induction, or a combination thereof. The heater may be connected to a pressurization device (e.g., a fan) that moves air into the heating chamber, the delivery chamber, or both. The heater may be a heating wire. The heater may be a positive temperature coefficient heater. The heater may be an induction heater. The heater may be an electric heater. The heater may heat through friction with the substance or two pieces within the heater. The heater may agitate the substance while heating the substance. The heater may agitate to create heat. The heater may be a ceramic heater. The heater may be a peltier device. The heater may increase pressure within the delivery chamber so that the substance is moved out of the delivery chamber, into the distribution member, into contact with a user, or a combination thereof. The heater may increase pressure by moving air into the delivery chamber. The heater may increase pressure my increasing a temperature within the delivery chamber. The heater may heat for a predetermined amount of time and then turn off once a predetermined heat is achieved, once heat is applied for a predetermined amount of time, or both. The heater may work in conjunction with a pressurization device.

The pressurization functions to create pressure within the delivery chamber, the heating chamber, or both. The pressurization device may create a positive pressure or a negative pressure. The pressurization device may push vapors through the heating chamber, the delivery chamber or both. The pressurization device may pull vapors through the heating chamber, the delivery chamber or both. The pressurization device may increase pressure with a blower or a fan. The pressurization device may apply heat in addition to the heater to increase pressure. The pressurization may actuate and reduce a volume so that pressure is increased. For example, after a predetermined amount of time the pressurization device may reduce an area within the heating chamber, the delivery chamber, or both so that vapor is applied. The heater, the pressurization device, or both may be controlled by one or more controllers that may be located within the heating chamber, a control chamber, or both.

The control chamber may function to control the delivery system, the delivery device, the heater, or a combination thereof. The control chamber may house one or more control elements that may function to control the delivery system, the delivery device, or both. The control chamber may house a battery, printed circuit board, controller, microcontroller, memory, one or more switches, motors, timers, processors, random access memory, flash memory, or a combination thereof. The control chamber may include one or more processors or microprocessors that control the delivery system or the delivery device. The control chamber may be electrically connected to the heating chamber. The control chamber may thermally protect the components therein from the heating chamber. The control chamber may be sealed. The control chamber may be thermally closed. The control chamber may be sealed from fluids, dust, debris, or a combination thereof. The control chamber may include one or more ports. The ports may be used to input information, charge the battery, set application times, or a combination thereof.

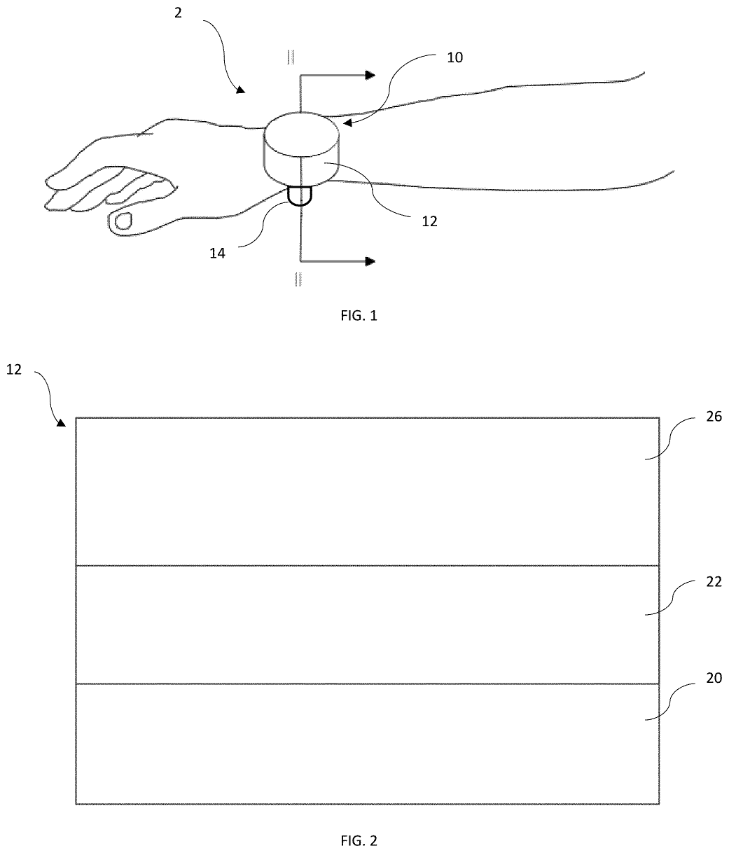

FIG. 1 illustrates a perspective view of the delivery system 2. The delivery system includes a delivery device 10 having a housing 12. The delivery device 10 is connected to a user via an attachment mechanism 14.

FIG. 2 is a cross-sectional view of the housing 12 of FIG. 1. The housing 12 includes a delivery chamber 20, a heating chamber 22, and a control chamber 26.

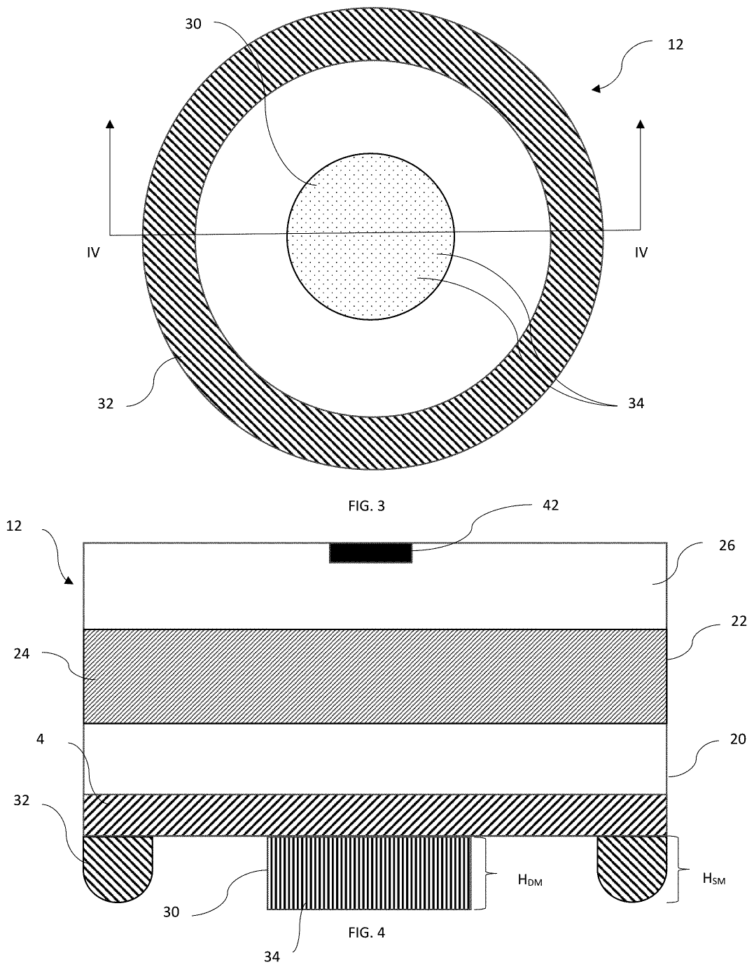

FIG. 3 is a bottom view of a housing 12. The housing 12 includes a distribution member 30 and a sealing member 32 that forms a seal between the housing 12 and a user (not shown). The distribution member 30 includes a plurality of perforations 34 that permit delivery of the substance to skin of a user.

FIG. 4 is a cross-sectional view of the housing 12 of FIG. 3. The housing 12 includes a delivery chamber 20, a heating chamber 22, and a control chamber 26. A bottom of the delivery chamber 20 includes a distribution member 30 that provides a substance 4 to a user (not shown). The substance 4 is liquified or vaporized by a heater 24 within the heating chamber 22 so that the liquid or vapor extend through the perforations 34 of the distribution member 30 into contact with the user. The liquid or vapor is sealed between the user and the housing 12 by a sealing member 32. The sealing member 32 has a height (H_{SM}) and a distribution member 30 has a height (H_{DM}). A pressurization device 42 assists in moving the vapor through the distribution member 30.

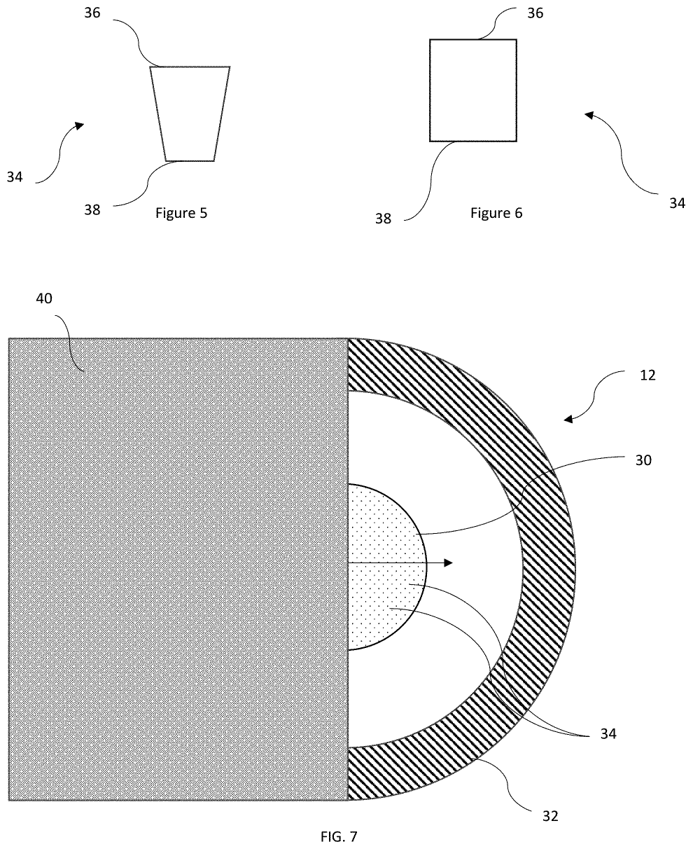

FIG. 5 is a cross-sectional view of a perforation 34 where an opening at top 36 is larger than an opening at the bottom 38.

FIG. 6 is a cross-sectional view of a perforation 34 where an opening at a top 36 and a bottom 38 are equal.

FIG. 7 is a view of a housing 12 of FIG. 3 from a bottom. The housing 12 includes a distribution member 30 and a sealing member 32 that forms a seal between the housing 12 and a user (not shown). The distribution member 30 includes a plurality of perforations 34 that permit delivery of the substance to skin of a user. A flap 40 is installed that is covering a portion of the area to limit an amount of distribution.

Variation 1 may comprise A transdermal delivery system comprising: a delivery device; and a band configured to connect the delivery device to a body part of a user, the delivery device comprising: a delivery chamber that is configured to receive a substance and a heating chamber configured to heat the substance in the delivery chamber so that the substance is liquified, vaporized, or both and exits the delivery chamber into contact with the body part of the user so that the substance is transdermally absorbed.

Variation 2 may include variation 1 and may further comprise a control chamber that is configured to control components within the heating chamber, control distribution of the substance, or both.

Variation 3 may include any of variations 1-2 and may further comprise: a heater located within the heating chamber.

Variation 4 may include any of variations 1-3 and may further comprise: an agitator, an ultrasonic vibration device, of both are located within the heating chamber to agitate the substance while the substance is being heated to facilitate movement of the substance.

Variation 5 may include any of variations 1-4 and may further comprise: a sealing member that forms a seal between the body part of the user and the delivery device.

Variation 6 may include any of variations 1-5 and may further comprise: a distribution member that assists in spreading the substance over an area.

Variation 7 may include any of variations 1-6 and may further comprise: the distribution member distributes the substance within an area that is substantially a same as the delivery device.

Variation 8 may include any of variations 1-7 and may further comprise: the sealing member is compressible.

Variation 9 may include any of variations 1-8 and may further comprise: the sealing member is elastomeric.

Variation 10 may include any of variations 1-9 and may further comprise: the heater vaporizes the substance so that some or all of the substance is turned into a gas and the gas is moved into contact with the body part of the user.

Variation 11 may include any of variations 1-10 and may further comprise: the heater liquifies some or all of the substance so that the liquified substance is moved into contact with the body part of the user.

Variation 12 may include any of variations 1-11 and may further comprise: the heater is a heating wire.

Variation 13 may include any of variations 1-12 and may further comprise: the distribution member includes a plurality of perforations that permit the substance to exit the distribution member.

Variation 14 may include any of variations 1-13 and may further comprise: the perforations are a channel or a through hole in the distribution member.

Variation 15 may include any of variations 1-14 and may further comprise: the perforations have larger opening at a top than at a bottom of the distribution member.

Variation 16 may include any of variations 1-15 and may further comprise: a flap that is movable to cover all or a portion of the perforations.

Variation 17 may include any of variations 1-16 and may further comprise: some or all of the perforations are conical in shape.

Variation 18 may include any of variations 1-17 and may further comprise: wherein the heater is a Peltier device.

Variation 19 may include any of variations 1-18 and may further comprise: a timer that allows a user to automatically turn on and off the device.

Variation 20 may include any of variations 1-19 and may further comprise: a pressurization device to assist in moving the substance is liquified, vaporized, or both into and through the delivery chamber.

Any numerical values recited herein include all values from the lower value to the upper value in increments of one unit provided that there is a separation of at least 2 units between any lower value and any higher value. As an example, if it is stated that the amount of a component or a value of a process variable such as, for example, temperature, pressure, time and the like is, for example, from 1 to 90, preferably from 20 to 80, more preferably from 30 to 70, it is intended that values such as 15 to 85, 22 to 68, 43 to 51, 30 to 32 etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Unless otherwise stated, all ranges include both endpoints and all numbers between the endpoints. The use of “about” or “approximately” in connection with a range applies to both ends of the range. Thus, “about 20 to 30” is intended to cover “about 20 to about 30”, inclusive of at least the specified endpoints.

The disclosures of all articles and references, including patent applications and publications, are incorporated by reference for all purposes. The term “consisting essentially of” to describe a combination shall include the elements, ingredients, components or steps identified, and such other elements ingredients, components or steps that do not materially affect the basic and novel characteristics of the combination. The use of the terms “comprising” or “including” to describe combinations of elements, ingredients, components or steps herein also contemplates embodiments that consist essentially of or even consists of the elements, ingredients, components or steps.

Plural elements, ingredients, components or steps can be provided by a single integrated element, ingredient, component or step. Alternatively, a single integrated element, ingredient, component or step might be divided into separate plural elements, ingredients, components or steps. The disclosure of “a” or “one” to describe an element, ingredient, component or step is not intended to foreclose additional elements, ingredients, components or steps.

It is understood that the above description is intended to be illustrative and not restrictive. Many embodiments as well as many applications besides the examples provided will be apparent to those of skill in the art upon reading the above description. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

### ELEMENT LIST

2 Transdermal Delivery System
4 Substance
10 Delivery Device
12 Housing
14 Attachment mechanism
20 Delivery Chamber
22 Heating Chamber
24 Heater
26 Control Chamber
30 Distribution Member
32 Sealing Member
34 Perforations
36 Top
38 Bottom
40 Flap
42 Pressurization Device

I claim:

1. A transdermal delivery system:
a delivery device; and
a band configured to connect the delivery device to a body part of a user, the delivery device comprising:
a delivery chamber that is configured to receive a substance and
a heating chamber configured to heat the substance in the delivery chamber so that the substance is liquified, vaporized, or both and exits the delivery chamber into contact with the body part of the user so that the substance is transdermally absorbed.

2. The transdermal delivery system of claim 1, further comprising a control chamber that is configured to control components within the heating chamber, control distribution of the substance, or both.

3. The transdermal delivery system of claim 1, further comprising a heater located within the heating chamber.

4. The transdermal delivery system of claim 1, further comprising an agitator, an ultrasonic vibration device, of both are located within the heating chamber to agitate the substance while the substance is being heated to facilitate movement of the substance.

5. The transdermal delivery system of claim 1, further comprising a sealing member that forms a seal between the body part of the user and the delivery device.

6. The transdermal delivery system of claim 1, further comprising a distribution member that assists in spreading the substance over an area.

7. The transdermal delivery system of claim 6, wherein the distribution member distributes the substance within an area that is substantially a same as the delivery device.

8. The transdermal delivery system of claim 5, wherein the sealing member is compressible.

9. The transdermal delivery system of claim 5, wherein the sealing member is elastomeric.

10. The transdermal delivery system of claim 3, wherein the heater vaporizes the substance so that some or all of the substance is turned into a gas and the gas is moved into contact with the body part of the user.

11. The transdermal delivery system of claim 3, wherein the heater liquifies some or all of the substance so that the liquified substance is moved into contact with the body part of the user.

12. The transdermal delivery system of claim 3, wherein the heater is a heating wire.

13. The transdermal delivery system of claim 6, wherein the distribution member includes a plurality of perforations that permit the substance to exit the distribution member.

14. The transdermal delivery system of claim 13, wherein the perforations are a channel or a through hole in the distribution member.

15. The transdermal delivery system of claim 13, wherein the perforations have larger opening at a top than at a bottom of the distribution member.

16. The transdermal delivery system of claim 13, further comprising a flap that is movable to cover all or a portion of the perforations.

17. The transdermal delivery system of claim 13, wherein some or all of the perforations are conical in shape.

18. The transdermal delivery system of claim 1, wherein the heater is a Peltier device.

19. The transdermal delivery system of claim 1, further comprising a timer that allows a user to automatically turn on and off the device.

20. The transdermal delivery system of claim 1, further comprising a pressurization device to assist in moving the substance is liquified, vaporized, or both into and through the delivery chamber.
